(19) United States
(12) Patent Application Publication
(10) Pub. No.: US 2025/0334498 A1
(43) Pub. Date: Oct. 30, 2025

(54) EMERGENT BEHAVIOR-BASED STRATEGIES FOR ENVIRONMENTAL PFAS REMEDIATION

(71) Applicant: GEO-RHEO, LLC, Sisters, OR (US)

(72) Inventor: Patrick Richard BRADY, Sisters, OR (US)

(21) Appl. No.: 18/646,515

(22) Filed: Apr. 25, 2024

(51) Int. Cl. G01N 13/02; A62D 3/40; A62D 101/22; G01N 1/38

(52) U.S. Cl. CPC G01N 13/02; A62D 3/40; G01N 1/38; A62D 2101/22

(57) ABSTRACT

Methods for characterizing an environmental contaminant containing a PFAS formulation. The methods involve obtaining environmental samples with the PFAS formulation, creating a dilution series of the PFAS formulation, determining the static surface tension for each dilution, and plotting the static surface tension against the logarithm of the PFAS formulation concentration to generate an emergent behavior curve. Utilizing this curve, PFAS formulation concentration can be assigned within non-emergent dispersive, weakly emergent, and strongly emergent behavior concentration ranges, provides a systematic and efficient method for determining environmental sites most likely to shed additional PFAS into the environment. The emergent behavior curve also permits total PFAS formulation concentration to be effectively measured in real time, and to be converted into total PFAS TOPs concentration.

### TECHNICAL FIELD

The present disclosure relates generally to environmental remediation, and more particularly to the characterization of per- and polyfluoroalkyl substances (PFAS) in contaminated media.

### BACKGROUND

Per- and polyfluoroalkyl substances (PFAS or PFASs) are a group of synthetic organofluorine compounds that include multiple fluorine atoms attached to a hydrocarbon chain. As a result of this composition, PFASs can exhibit many desirable properties for industrial applications, including enhanced chemical stability and water-resistance. Fluorinated surfactants, in particular, have been found to be much more effective at reducing the surface tension of water than comparable hydrocarbon surfactants, and so have found widespread industrial use.

Unfortunately, the increasing prevalence of PFASs has resulted in an increasing release of PFASs into the environment, where their chemical stability and resistance to decomposition by natural processes have led them to be labeled “forever chemicals”. Only recently have the extent of the environmental impact and the toxicity of PFASs begun to be studied in depth, but PFAS exposure has already been linked to increased risk of dyslipidemia (abnormally high cholesterol), suboptimal antibody response, reduced infant and fetal growth, and higher rates of kidney cancer, among other health effects. The manufacture and use of PFASs has therefore come under increased scrutiny and regulation, in the US and other countries.

Environmental regulation has historically focused on the behavior of individual chemicals, their toxicity, and characteristic properties. An exception is regulation of non-aqueous phase liquids, including petroleum products such as gasoline and diesel. These formulations include a range of individual hydrocarbon compounds and additives, and are considered to be Light Non-Aqueous Phase Liquids (LNAPLs). When released to the environment these LNAPLs typically occur at the top of the capillary fringe above the water table, where they can be measured easily. Investigation and remediation of a chemical or petroleum fuel release is assessed by analyzing samples and comparing the results to a numeric standard.

This approach is consistent with the nature of the classic contaminants released to the environment. For example, gasoline contains a maximum of 3% additives with the remaining mass being various hydrocarbons. Remedial efforts for spilled gasoline can be evaluated through an analysis of all hydrocarbons that correspond to those that fall within the gasoline hydrocarbon range. The entire mixture of gasoline related compounds can be measured, and the behavior of those compounds in the environment is well understood.

However, PFASs are typically released into the environment as part of a colloidal formulation, and not as an individual PFAS compound. These PFAS-stabilized formulations are complex systems, with the amount of PFAS surfactant that creates the formulation being generally less than 1% of the mass of the entire formulation. These colloidal formulations are “surfactant-water-oil” systems, as opposed to simple mixtures of surfactants dissolved in water. Furthermore, the properties and behaviors of the PFAS formulation are dramatically different from those of either water or a pure PFAS. Any evaluation and treatment of a PFAS formulation in the environment that is predicated upon the behavior of the PFAS component, rather than the properties of the formulation as a whole, will be flawed and inefficient, and potentially ineffective.

What is needed is a new paradigm for the evaluation and treatment of PFASs in the environment based upon the unique properties of the PFAS formulation, and not just the properties of its individual components.

### SUMMARY

The present disclosure is directed to methods of characterizing the presence or behavior of a PFAS formulation, and in particular a methodology of characterizing PFAS contamination of a site of interest using emergent behavior-based strategies.

In some aspects, the techniques described herein relate to a method of characterizing a PFAS formulation, including: obtaining one or more samples including a PFAS formulation; preparing a dilution series of the PFAS formulation; determining a static surface tension for each member of the dilution series of the PFAS formulation; plotting the determined static surface tension for each member of the dilution series versus a logarithm of a concentration of the PFAS formulation to create an emergent behavior curve; and using the emergent behavior curve, assigning a PFAS formulation concentration range for each of a non-emergent dispersive concentration range, a weakly emergent concentration range, and a strongly emergent behavior concentration range.

In some aspects, the techniques described herein relate to a method of characterizing an environmental site contaminated with a PFAS formulation, including: obtaining one or more samples at a plurality of locations within the environmental site contaminated with the PFAS formulation; preparing a dilution series of the PFAS formulation; measuring a static surface tension for each member of the dilution series of the PFAS formulation; plotting the measured static surface tension for each member of the dilution series versus a logarithm of a concentration of the PFAS formulation to create an emergent behavior curve; using the emergent behavior curve, assigning a PFAS formulation concentration range for each of a non-emergent dispersive concentration range, a weakly emergent concentration range, and a strongly emergent behavior concentration range; measuring a static surface tension for each of the one or more obtained samples; and using the emergent behavior curve to estimate an environmental PFAS formulation concentration at each of the plurality of locations where the one or more samples was obtained.

In some aspects, the techniques described herein relate to a method of at least partially remediating a site contaminated with a PFAS formulation, including: collecting a sample of the PFAS formulation at the site; preparing a dilution series of the PFAS formulation; measuring a static surface tension for each member of the dilution series of the PFAS formulation; plotting the measured static surface tension for each member of the dilution series versus a logarithm of a concentration of the PFAS formulation to create an emergent behavior curve; measuring a static surface tension for each of a plurality of samples collected at known locations within the site contaminated with the PFAS formulation; estimating, using the emergent behavior curve, a PFAS formulation concentration at each of the known locations within the site contaminated with the PFAS formulation; determining, using the emergent behavior curve, whether the PFAS formulation at each of the known locations within the site contaminated with the PFAS formulation is likely to shed additional PFAS into an environment of that location; and removing contaminated material from the site contaminated with the PFAS formulation at the known locations within the site where the environmental PFAS formulation concentrations are determined to be likely to shed additional PFAS into the environment of that location.

### BRIEF DESCRIPTION OF THE DRAWINGS

FIG. 1 is a diagram showing the use of AFFF firefighting foam, and its entry into the environment.

FIG. 2 is an exemplary emergent behavior curve for a PFAS formulation, showing a relationship between static surface tension and PFAS formulation concentration.

FIG. 3 is a semi-schematic depiction of PFAS molecule behavior within the non-emergent dispersive concentration range.

FIG. 4 is a semi-schematic depiction of PFAS molecule behavior within the weakly emergent concentration range.

FIG. 5 is a semi-schematic depiction of PFAS molecule behavior within the strongly emergent concentration range.

FIG. 6 is a representative illustration of a map of estimated PFAS contaminant concentration at an environmental site, obtained using an emergent behavior plot of the present disclosure.

FIG. 7 is a flowchart of an illustrative method according to the present disclosure.

FIG. 8 is a flowchart of another illustrative method according to the present disclosure.

FIG. 9 is a flowchart of yet another illustrative method according to the present disclosure.

### DETAILED DESCRIPTION

When trying to understand or predict the behavior of a system, even a complex system containing many components, it is often assumed that it will behave as if it is simply a sum of its component parts. That is, the properties of such a system will be related in a more or less linear fashion to the properties of the individual components.

In contrast, some complex systems are said to exhibit emergence, or emergent behavior, where the behavior or properties of the system cannot be easily predicted (in weakly emergent systems), or cannot be predicted at all (in strongly emergent systems), based upon knowledge of the individual components of the system. In such an emergent system, the behavior of system does not depend on its individual components, but on relationships, synergies, and interactions between those components. Put another way, the behavior of an emergent system is greater than the sum of its parts. As a result, emergent behavior cannot be predicted in a conventional linear fashion through the examination of an emergent system's individual components.

PFAS-stabilized formulations are emergent systems that exhibit non-linear behavior that changes depending on the concentration of the PFAS blend in the formulation. PFAS-stabilized microemulsions are clear, thermodynamically-stable isotropic liquid mixtures of oil, water, and surfactant, frequently present in combination with a cosurfactant. Such formulations spontaneously form microemulsions (droplet size 1 nm to 300 nm).

PFAS-stabilized formulations are often used to form aqueous film forming foams (AFFF) used in firefighting. The addition of a PFAS surfactant to the aqueous system lowers the surface tension of the water, which assists in the wetting and saturation properties of the resulting foam. An applied AFFF layer cools the fire and coats the fuel, starving it of oxygen, and will even self-heal if the foam is disrupted, and continue to coat the fuel.

These PFAS-stabilized formulations behave as non-Newtonian fluids having pseudoplastic and thixotropic properties. When subjected to increasing shear stress, the viscosity of the fluid is reduced, but recoils over time when the shear stresses are removed. AFFF foams are generated with the application of shear stress when the formulation is added to a charged aspirating fire nozzle.

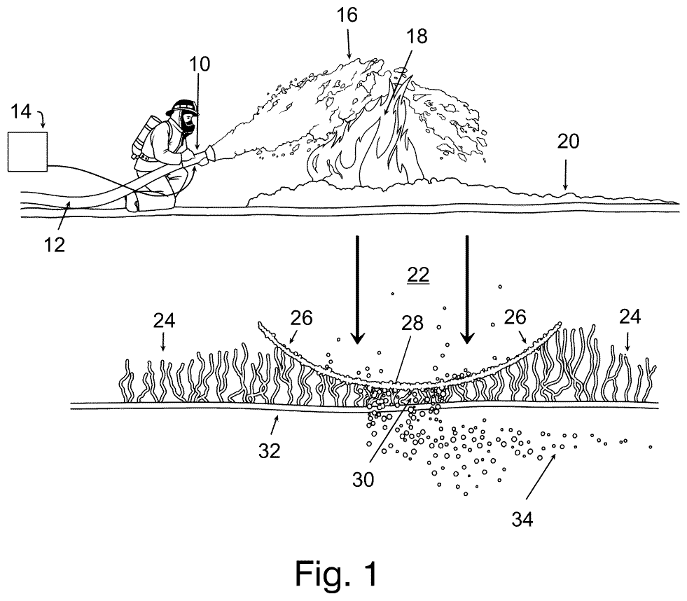

FIG. 1 depicts a typical AFFF foam firefighting arrangement. A fire nozzle 10 combines a large volume stream of high velocity/high pressure water 12 with AFFF formulation concentrate 14 at the design specifications, which cause multiple phenomena to occur almost instantaneously. The activation shear point of the AFF formulation is reached when the formulation contacts the water stream, substantially reducing its viscosity. A foam 16 is immediately formed at the exit of the firehose nozzle 10, which is then broadcast over a liquid hydrocarbon fire 18 to extinguish the fire.

After discharge the AFFF foam eventually decays over time (i.e., the bubbles of the foam collapse), generating a wastewater/formulation mixture 20 that then migrates into the subsoil 22 modifying the wetting kinetics of the subsoil along its downward path. Eventually, the mixture encounters the top of the capillary fringe 24 of the water table. The mixture increases its viscosity over time due to its thixotropic nature, and reconstitutes back to its original viscosity. Due to the decreased surface tension of the wastewater mixture, the capillary fringe is depressed, forming a localized depression 26 where a the PFAS mixture 28 will accumulate as a thin film.

Studies have shown that AFFF in wastewater can be well above its critical micelle concentration (CMC), a point at which a surfactant saturates a surface of a liquid and begins to form micelles and other complex structures in the bulk of the liquid. Concentrations above the CMC support the formation of a microemulsion, and the shedding of PFAS across the residual capillary fringe 30 into the water table 32, thereby acting as a long-term source of PFAS contamination as a large dilute plume of PFAS and other compounds 34, which can travel for miles. Bayesian inference studies have found that these source structures can shed PFAS to groundwater for hundreds of years.

By plotting the static surface tension of a PFAS-containing solution as a function of the logarithm of the PFAS formulation concentration, the complex behaviors exhibited by such mixtures can be revealed. A sample of the PFAS formulation of interest is obtained, and serially diluted in distilled water to create the desired range of concentrations. The static surface tension can be measured using any appropriate instrumentation or methodology, such as for example by using a Wilhelmy plate apparatus or Du Noüy ring apparatus. Static surface tension can be determined by making contact angle measurements for each prepared concentration. While a Wilhelmy plate apparatus can measure advancing, receding and hysteresis contact angles, it may be particularly advantageous to determine contact angle measurement using methods described in US patent publication no. 2022/0307961, published Sep. 29, 2022, hereby incorporated by reference.

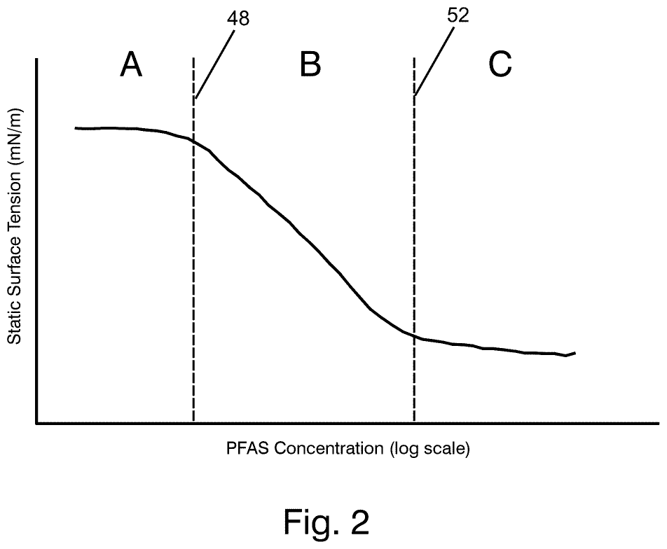

The resulting plot, as shown in FIG. 2, may be referred to as an emergent behavior curve. Alternatively, it may be referred to as Gibbs adsorption isotherm plot for multicomponent systems, or a critical micelle concentration plot. The emergent behavior curve exhibits three distinct regions corresponding to different behaviors of the PFAS-stabilized formulation as the concentration of PFAS in the formulation changes. The differences in static surface tension reflect how physical properties of the PFAS-stabilized formulations shift from exhibiting a linear response to a non-linear response with increasing PFAS formulation concentration.

Such self-aggregated PFAS microemulsions are emergent systems. A system is said to display emergent behavior when it exhibits properties or behaviors that are not exhibited by, or predictable from, the individual components of the system. Put another way, the properties of an emergent systems can be distinctly different from the individual properties of its constituent components.

Referring to FIG. 2, the region A corresponds to measurements made using lower concentrations of PFAS. At these concentrations the amount of PFAS present in the mixture is insufficient to promote self-aggregation of the PFAS molecules, and the properties of the formulation can behave in a linear fashion, and with static surface tension gradually decreasing as PFAS formulation concentration increases. In this region, the dynamic surface tension of the mixture does not change with changes in concentration, and the fluid behaves as a Newtonian fluid. Typically, the PFAS formulation concentrations for region A correspond to a value below the median lethal concentration, or LC₅₀, of that PFAS formulation, where LC₅₀ (or LD₅₀, the median lethal dose) corresponds to the dose required to kill half the members of a tested population after a specified test duration.

Where the static surface tension measured for a localized environmental sample indicates that PFAS formulation concentration at that location falls within region A of the calibration curve, the PFAS present at that location will not exhibit spontaneous self-assembly, or form more complex structures existing as dispersed PFAS. More significantly, the environmental PFAS in that concentration present at that location will not shed additional PFAS into the groundwater, or form a PFAS plume extending downstream from that location. As the behavior of the formulations in region A behave in like a Newtonian fluid, region A of the plot of FIG. 2 may be referred to as the “non-emergent dispersive plateau”. The PFAS formulation concentration range corresponding to the non-emergent plateau can be referred to as the non-emergent dispersive concentration range.

As PFAS formulation concentration increases, there is a steep decrease in static surface tension in region B of FIG. 2. In this region, the static surface tension of the PFAS-stabilized formulations depends upon PFAS formulation concentration as a power law function. That is, the plot of static surface tension varies as the logarithm of the concentration, resulting in an overall linearity in region B, which may be referred to as a “power law region.” The power law relationship defines a region where a small change in one variable results in a large change in the behavior of the system. The PFAS formulation concentration range corresponding to the power law region can be referred to as the weakly emergent concentration range.

Within region B, as PFAS formulation concentration increases, the dynamic surface tension in the bulk of the PFAS-stabilized formulation begins to decrease with the static surface tension, and the mixture exhibits non-Newtonian behavior. In this region laminar structures can form spontaneously, indicating that the creation of such aggregates is energetically favored. PFAS toxicity for solutions having a concentration within region B are above the median lethal concentration (LD₅₀) but below the lethal concentration (LC or LC₁₀₀) for that PFAS formulation. In this region, autopolymerization of PFAS takes place, resulting in the formation of microemulsions. The spontaneous creation of such PFAS aggregates results in the shedding of PFAS from the contaminating PFAS formulations into the water table, creating PFAS plumes downstream that can extend for miles.

AS PFAS formulation concentration increases still further, the plot of FIG. 2 enters region C, or the “strongly emergent plateau”. The PFAS formulation concentration range corresponding to the strongly emergent plateau can be referred to as the strongly emergent behavior concentration range.

PFAS formulation concentration levels in region C begin at the critical micelle concentration (CMC), and remains above the lethal concentration (LC). The PFAS-stabilized formulation exhibits the formation of PFAS-stabilized micelles in the bulk of the mixture. As the surface of the formulation in this region is completely saturated with PFAS, there is substantially no further decrease in static surface tension as concentration increases further, although dynamic surface tension can substantially decrease in this region. The formulation exhibits non-Newtonian behavior, and the PFAS molecules spontaneously form micelles as well as regions of liquid crystal structures. At these concentrations the PFAS-stabilized formulations present in the environment will freely shed PFAS into the water table and create plumes of additional PFAS contamination. Environmental PFAS that is present at strongly emergent concentrations is also prone to vertical migration due to the Marangoni effect.

### The PFAS Formulation

As used herein, the “PFAS formulation” refers to a chemical compound, or mixture of chemical compounds, including at least one per- or polyfluoroalkylated substance, present in an aqueous sample. Typically, PFAS formulation refers to the PFAS or PFAS-containing portion of a solution or mixture, such as an aqueous sample, collected sample, or a manufactured sample, that exhibits emergent behavior as discussed above due to the presence of the PFAS formulation. That is, where a sample is determined to include a PFAS component, at least that portion of the sample corresponding to PFAS is termed the PFAS formulation.

Where a manufactured sample is prepared by the addition of one or more known or unknown PFAS compounds to an aqueous solution, the identity of the PFAS compound or compounds in the PFAS formulation is obviously known. Where a sample is collected that includes a PFAS formulation, it may be possible to identify the PFAS formulation present in the collected sample by virtue of having knowledge of a prior PFAS release at or near the site where the sample was collected, such as for example AFFF firefighting foam formulations.

Where a collected sample includes a PFAS formulation, and there is no record of the identity of the origin of the PFAS at the site of interest where the sample is collected, it may be necessary to search for and collect a sample at that site that exhibits the highest PFAS formulation concentration available, so that either the PFAS formulation can be characterized by analysis of the collected sample by known methods, or the collected sample itself can be used to prepare a calibration curve, as will be discussed below.

Where a dilution series cannot be readily prepared using a collected sample as the source of the PFAS formulation, for example where it is difficult or impossible to obtain samples having a sufficiently high concentration of PFAS in the environment, it may be helpful, or even necessary, to prepare higher concentration calibration solutions using an alternative source of PFAS. The alternative source of PFAS should be selected so as to create a solution that corresponds as closely as possible to those observed for the collected PFAS formulation.

### PFAS-Containing Sample

The presence and/or concentration of PFAS at a given location is typically determined or verified by sampling at that location. Any location that includes or is suspected to include a PFAS formulation is an appropriate location for the purposes of this disclosure. A sample obtained at such a location may be any combination of fluids and solids, including aqueous solutions, colloidal mixtures, semi-solid materials, and solid materials, among others.

The obtained sample may be or include an environmental sample. That is, the sample may be collected at a site of interest that may be contaminated by PFAS, either directly, or by the migration of PFAS via underground water movement. Alternatively, the obtained sample may be or include a waste sample, where the sample may be obtained from any stage of a waste disposal or containment process. For example, a waste sample may be obtained from untreated waste materials, in order to evaluate the PFAS content of those waste materials; a waste sample may be obtained from treated waste materials, in order to evaluate the efficacy of a treatment for removing or remediating PFAS in those materials; and a waste sample may be obtained from a waste disposal site, such as a hazardous waste disposal site, in order evaluate the PFAS content of the hazardous materials located at the site; among other possibilities.

An obtained sample is typically collected in situ at a location or site of interest, such as an environmental location where contamination by PFAS is suspected, or a hazardous waste facility. Typically, a plurality of samples is collected at such a location, and the location where each sample was collected is recorded. The sample can be a fluid sample, a colloidal sample, a semi-solid sample, or a solid sample, without limitation. Where the environmental sample is or includes a fluid, the fluid is typically but not exclusively an aqueous fluid. The fluid sample can be analyzed directly, or can be subject to one or more purification steps, such as for example filtration, prior to analysis.

Where the environmental sample includes a fluid, the source of that fluid may be groundwater, pore water, perched water, excavation water, surface water, or leachate, among others sources of water. The environmental sample may be collected from a surface body of water or from ground water, for example from a well, from a borehole, and/or from the leachate from a waste facility. Where the sample is collected from a well, the well may be a monitoring well, a water supply well, or any other type of well. Where the sample is collected from a borehole, the borehole may be formed by direct-push drilling, or any other suitable drilling method.

Where an obtained sample is or includes a solid, the sample may be combined with an appropriate liquid, and the resulting fluid may then be analyzed directly as a fluid sample, or the sample may be subjected to one or more purification steps prior to analysis as a fluid sample. Where the sample is being combined with an appropriate fluid, it can be immersed in the fluid, shaken with the fluid, dissolved in the fluid, or placed in contact with the fluid using any other appropriate method. Typically, the sample is combined with water, particularly distilled water, however any other relatively polar solvent may be used to extract a PFAS formulation of interest from an environmental sample, including for example methanol or ethanol. In one aspect of the present disclosure, a fluid sample may be obtained by simply pouring water over a surface of interest, collecting the water, and testing the water as a fluid sample.

Where an obtained sample is or includes a solid, the solid may be or include a soil sample a porous media sample, or a colloidal media sample, among others. A solid environmental sample may be collected from any suitable location or source, including but not limited to soil borings, boreholes (including direct-push drill boreholes), surface soils, waste streams, or any suitable excavation.

### Surface Energy Footprint

As discussed above, the emergent behavior curve for a given PFAS formulation can be prepared by plotting the surface energy characteristics of the PFAS formulation as a function of the logarithm of the concentration of the PFAS formulation. Although the surface energy characteristics of a fluid sample may be readily determined using any of a variety of known methods and instruments, it is typically most straightforward to determine a surface energy footprint for a fluid sample by the measurement of surface tension.

For example, static surface tension may be determined using the Wilhelmy plate or Du Nouy ring method, while dynamic surface tension may be determined using the bubble pressure method, among others. More particularly, however, surface tension may be determined via contact angle measurement.

A contact angle is the angle defined by a liquid-vapor interface where a drop of liquid meets a solid surface, and reflects the relative strength of the interactions between the liquid, the solid, and the vapor. Where a liquid has a high degree of surface tension, such as water, and the surface is relatively nonpolar, measured contact angles can be very high. Alternatively, a drop of nonpolar liquid on a nonpolar surface will typically spread out, exhibiting a very low contact angle.

Amphiphilic compounds, such as surfactants, have a tendency to undergo self-assembly on high energy surfaces, due largely to Coulombic interactions, and can create ordered layers or films on the surface. The surface energy of a given surface will decrease or increase and exhibit altered characteristics (changes in relative polar and dispersive portions of total surface energy) after being covered by such self-assembled amphiphilic structures.

A preferred surface energy measurement therefore involves measuring the surface energy of a clean substrate having relatively high surface energy by measurement of contact angles at that surface, followed by exposing the substrate to a fluid sample of interest. If amphiphilic compounds are present in the fluid sample, such as PFAS, then they should spontaneously adsorb onto the high energy surface of the substrate, and change the surface energy characteristics of the substrate surface. Contact angle measurement of the substrate surface before and after such exposure can therefore provide qualitative or quantitative information about the presence, amount, and even the type of PFAS formulation from which the fluid sample was derived.

Although any contact angle measuring device can be used for such determinations, it is preferable that the contact angle measurement apparatus be relatively small and lightweight, so that it can be readily used in the field. An exemplary contact angle measurement apparatus useful in the context of the present disclosure was described by Friedrich et al. in U.S. Pat. No. 9,816,909 (hereby incorporated by reference for all purposes).

### Emergent Behavior Calibration Curve

As discussed above, for a given PFAS formulation the preparation of a dilution series and the determination of static surface tension for the members of that dilution series can be used to prepare an emergent behavior curve, as shown in FIG. 2. The emergent behavior curve may be prepared by plotting measured static surface tension for a member of the dilution series versus the logarithm of the concentration of the PFAS formulation in that member of the dilution series. Once the emergent behavior curve is created, the emergent behavior curve may be divided into three PFAS formulation concentration ranges corresponding to a non-emergent dispersive concentration range (region A), a weakly emergent concentration range (region B), and a strongly emergent behavior concentration range (region C).

The emergent behavior curve may be readily divided into regions A, B, and C based upon the characteristic shape of the curve. Typically, the emergent behavior curve will exhibit a plateau at low PFAS formulation concentrations, corresponding to region A, and a plateau at higher PFAS formulation concentrations, corresponding to region C. Between the two plateaus, the emergent behavior curve typically exhibits a substantially linear region, corresponding to region B. The concentration range for each of regions A, B, and C may therefore be assigned qualitatively based simply upon the shape of the emergent behavior curve.

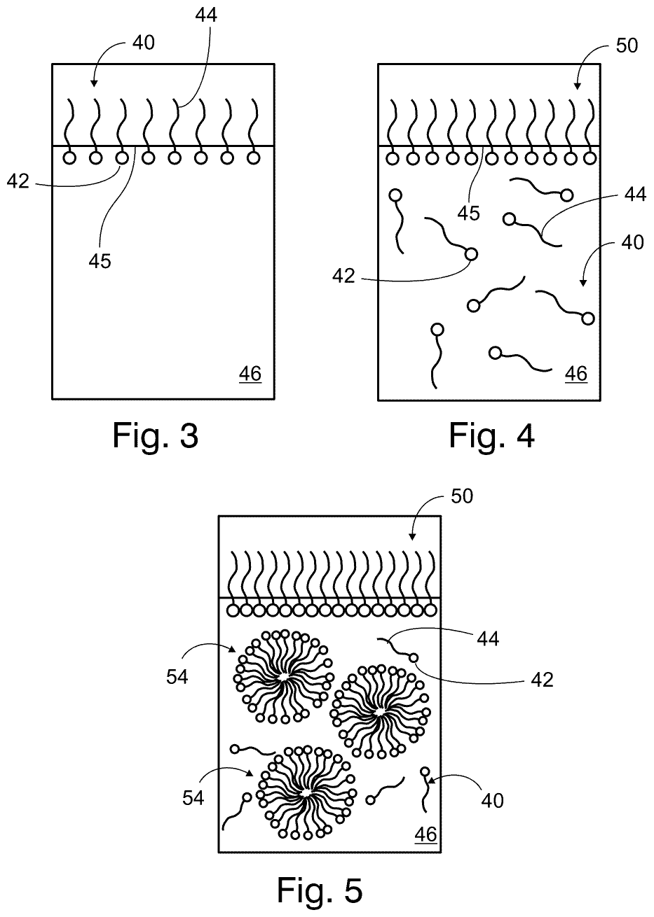

At concentrations within region A, or the non-emergent dispersive region, PFAS is present in solution at a sufficiently low concentration that the PFAS does not substantially self-aggregate, as shown in FIG. 3. Although PFAS surfactant molecules 40, including a polar hydrophilic head 42 and a non-polar hydrophobic tail 44, may begin to self-order at the surface 45 of aqueous solution 46, the PFAS molecules are not substantially present in the bulk of the solution.

The boundary between the non-emergent dispersive plateau of region A and the weakly emergent range of region B may be referred to as a limit of emergence 48, as shown in FIG. 2, which represents a PFAS formulation concentration at which the molecules of the PFAS components present in the fluid begin to exhibit synergistic interactions between each other, as reflected by the changes in the measured static surface tension. As shown in FIG. 4, within the weakly emergent concentration range of region B, PFAS molecules 40 typically form discontinuous ordered layers 50 at the solution surface 45, and PFAS molecules begin to appear within the bulk of solution 46. As the PFAS interactions become more substantial at concentrations within region B, or the weakly emergent concentration range, changes in PFAS formulation concentration begin to result in more dramatic changes in measured static surface tension. Static surface tension can be said to exhibit a “power law” relationship with PFAS formulation concentration, as static surface tension varies with the logarithm of PFAS formulation concentration. In this region, a small change in PFAS formulation concentration results in a much greater effective change in static surface tension.

Put another way, with increased PFAS formulation concentration, the complexity of the self-organizing structures in the system increases. With increasing concentration, layers of PFAS molecules may begin to interact to form micelles, and then at higher concentrations the micelles may act as building blocks to form more complex structures. At relatively high concentrations of micelles, the system may form regions of liquid crystals. When present in the environment, as the complexity of micellar structures in a composition increases, so does the potential of the PFAS formulation to migrate vertically.

The boundary between the weakly emergent concentration range of region B and the strongly emergent concentration range of region C may be referred to as the critical micelle concentration (CMC) 52. Beyond the critical micelle concentration of the PFAS formulation, within region C, the intermolecular interactions of PFAS component molecules substantially increase. As shown in FIG. 5, the PFAS molecules 40 spontaneously form self-aggregated micelle structures 54, foaming behavior becomes more marked, and the impact of changes in the concentration of the PFAS components of the solution do not effect static surface tension measurements as strongly.

The boundaries between regions A, B, and C may be identified using more quantitative methods. For example, the boundaries between the concentration ranges reflected by regions A, B, and C may be assigned by evaluating a rate of change of the instantaneous slope of the emergent behavior curve itself. In a typical emergent behavior curve, the shape of the curve will result in the rate of change of the slope exhibiting two maxima, corresponding to the limit of emergence 48 and the critical micelle concentration 52. The rate of change of the slope of the curve can therefore be used to determine the boundaries between regions A, B, and C of the emergent behavior curve.

Alternatively, or in addition, a mathematical analysis of the emergent behavior curve may be used to identify those portions of the curve that may exhibit a higher degree of linearity. For example, the weakly emergent concentration region B of the emergent behavior curve typically greater linearity than either the non-emergent dispersive plateau of region A or the strongly emergent behavior plateau of region C. The weakly emergent (or power law) concentration range can therefore be assigned as that portion of the central region of the emergent behavior curve that exhibits the closest fit to a straight line, while the portions of the curve on either side of that linear region can be assigned to the non-emergent dispersive concentration range and the strongly emergent behavior concentration range, respectively.

Once the emergent behavior curve has been prepared, and the non-emergent dispersive concentration range, the weakly emergent concentration range, and the strongly emergent behavior concentration range have been determined, the emergent behavior curve may then be used to characterize and/or evaluate the PFAS content of obtained samples, as well as predicting the future behavior of PFAS-containing mixtures based upon their PFAS formulation concentrations, as will be discussed in greater detail below.

### Total Oxidizable Precursor (TOP) Analysis

Current conventional methods of analyzing environmental PFAS contamination fail to consider the non-Newtonian behavior of PFAS-stabilized mixtures, and typically under-report the amount of PFAS present in the environment. The best such method includes a determination of Total Oxidizable Precursor (TOP) in samples, and includes oxidizing the sample, liberating the PFAS compound from their colloidal formulations, and removing elements of unsaturation in the PFAS components to convert them into fully saturated PFAS for measurement. TOPs analysis normally shows significantly higher concentration of total PFAS than other analytical methods used to characterize samples containing PFAS.

Currently, without performing an analysis of TOPs data the full extent of PFAS present at a contamination site cannot be accurately measured. More problematically, the effectiveness of any ongoing efforts to remove PFAS from the site cannot be evaluated effectively without performing an analysis for TOPs in treated materials. Unfortunately, TOPs analysis can require a turnaround time of three to six weeks and the use of an off-site commercial laboratory, resulting in delays and dramatically increased expense. In addition, a TOPs analysis will only provide a measure of PFAS concentration, and not an indication of PFAS formulation concentration.

By employing an emergent behavior calibration curve, the impact of the full blend of PFAS present in a sample, or the PFAS formulation concentration, may be effectively measured in real time, and the PFAS formulation concentration can be converted into a total PFAS TOPs concentration by performing a TOPs analysis of the PFAS formulation used to prepare the dilution series for the emergent behavior curve, and then proportioning out the total PFAS TOPs results across the curve.

For example, the dilution series used to prepare an emergent behavior curve may be analyzed to determine a corresponding PFAS TOPS concentration using a certified laboratory. The emergent behavior curve can then be effectively used to estimate total PFAS TOPs concentration in obtained samples from the field. Particularly where an obtained field sample is a fluid, a simple measurement of static surface energy of the sample fluid allows for a rapid estimation of the surface excess/surface concentration of PFAS at the sample site. Surface excess/surface concentration is typically estimated to be 85%-89% of the total PFAS TOPs mass, with the balance estimated for the bulk concentration.

Obtained samples that include or consist of solid, semi-solid, porous, or colloidal materials may require extraction in order to generate a fluid sample for a total PFAS TOPs concentration estimation. A fluid sample may be generated by mixing a mass of distilled water equal to the mass of the field sample with the field sample. The static surface tension of the resulting leachate may then be measured and compared to the emergent behavior calibration curve in order to obtain an estimated PFAS TOPs for both the surface excess/surface concentration and the bulk material.

Total PFAS TOPs concentrations for field samples consisting of solid, semi-solid, porous, or colloidal matter can also be derived from sample leachate data by a generic or site-specific derived Dilution Attenuation Factor (DAF). Multiplying the TOP value of the bulk of the sample leachate by the appropriate DAF results in an estimation of total PFAS TOPs associated with the sampled media.

Compared to existing methods, the determination of PFAS TOPs from soil leachate data is a substantially more accurate and more efficient method of determining soil cleanup effectiveness, as cleanup standards are based on the ability of soils to hold contaminants in place. Soil leachate data therefore provides a strong measurement of a soil's capacity to hold PFAS.

### Removal and Remediation

Effective PFAS remedial strategies allow for immediate interim remedial actions that address active spreading or discharging source areas. Such interim actions are made substantially more effective and/or efficient when it becomes possible to field screen for PFAS contaminant presence and concentration in real time, and to determine the most mobile portion of a PFAS source area. In addition, disposal classification and treatment are important considerations for a successful remedial effort. Given the significant differences in behavior between the traditional chemical or petroleum contamination and PFAS-stabilized mixtures, a paradigm shift is necessary to facilitate a successful remedial outcome.

By considering the relationship between emergent behavior and PFAS formulation concentration, the disclosed methods may facilitate the estimation of total PFAS blend concentrations in real time, permit mapping the extent of PFAS and emergent media to provide a remedial target for removal, and aid in classifying PFAS remediation waste as either suitable for disposal at a non-hazardous waste landfill or requiring disposal at a hazardous waste landfill. Perhaps most significantly, sampling and evaluation of treated materials permits the remediation strategies being used to be evaluated for their effectiveness, in real time, thereby permitting decisions regarding immediate retreatment to be made in the field.

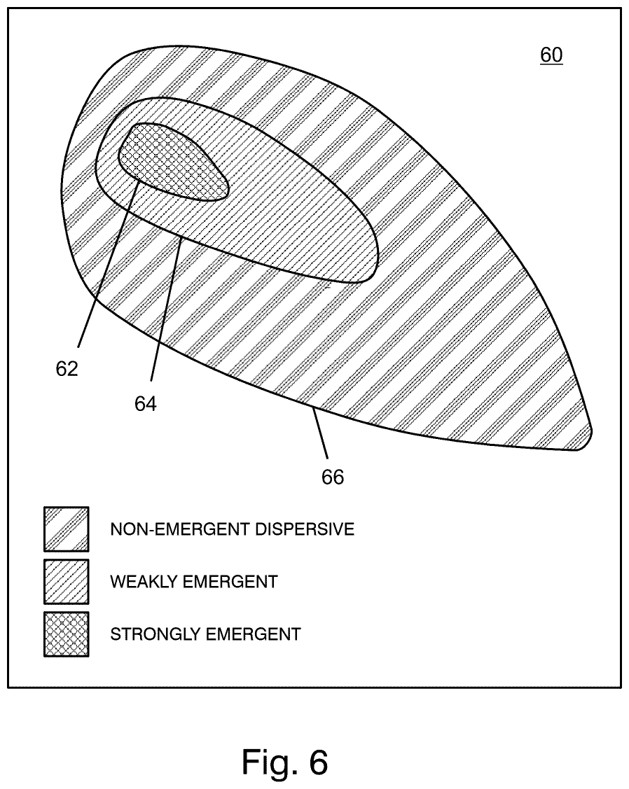

For example, once the emergent behavior calibration curve has been established for a PFAS formulation present at a selected contaminated site, analysis of field samples can be quickly used to map the estimated PFAS formulation concentrations within the site. FIG. 6 depicts an illustrative map 60 of a site of PFAS contamination where PFAS formulation concentrations have been determined by sampling across the site. A map of those concentrations is shown, with region 62 corresponding to an environmental presence of strongly emergent concentrations of the PFAS formulation, region 64 corresponding to an environmental presence of weakly emergent concentrations of the PFAS formulation, and region 66 corresponding to an environmental presence of non-emergent dispersive concentrations of the PFAS formulation. It should be appreciated that in addition to the characterization and evaluation of environmental sites, waste containment facilities, or similar disposal sites, can be similarly mapped for evaluation of PFAS formulation concentrations present at that location.

Although map 60 of FIG. 6 shows regions of PFAS formulation concentrations in two dimensions, it should be appreciated that such concentration data may more usefully be depicted in three dimensions, including an indication of depth below the surface of the ground as well as a location in the horizontal plane. For example, the vertical migration of the PFAS formulation would be expected to be greatest in region 62, where strongly emergent concentrations of the PFAS formulation are found, than in other areas of the site.

Such maps may then be used to determine which materials will be removed and remediated. For example, since PFAS compositions present in both the weakly emergent and strongly emergent concentration ranges will shed additional PFAS into the environment, it may be determined that materials in regions 62 and 64 should be removed and subjected to treatment/remediation. As the PFAS compositions in region 66 are not present at concentrations that will be subject to additional PFAS shedding, those materials may be left in place. Once the materials containing emergent concentrations of PFAS are removed, the remaining non-emergent dispersive concentrations of PFAS will naturally disperse and become even further diluted, to an extent that their concentration is below a level that requires remediation. In this way the remediation process shifts to a passive phase, where natural attenuation of the PFAS can take place, providing a significant advantage when compared to remediation strategies that do not include evaluating contaminated sites for emergent behavior.

Where removed materials are to be remediated, any accepted and useful method of remediating the PFAS-contaminated materials may be used, and the efficacy of the treatment evaluated using the emergent behavior calibration curve. The method of remediation selected should reduce the concentration of PFAS in the materials to at most a concentration that is below the power law range, and preferably much lower.

Remediation strategies that employ thermal volatilization using heated gases may be particularly useful for removing PFAS from collected PFAS-containing materials, as such methods can non-destructively remove, and collect, the PFAS present in porous and colloidal media. These remediation strategies further offer the advantage of being able to be conducted in the field, at the site of contamination. Such thermal volatilization strategies, methods, and apparatus are disclosed in U.S. Pat. No. 10,875,062 to Brady, U.S. Pat. No. 11,413,668 to Brady, and U.S. Pat. No. 11,484,922 to Brady, each of which is hereby incorporated by reference for all purposes.

### Methods

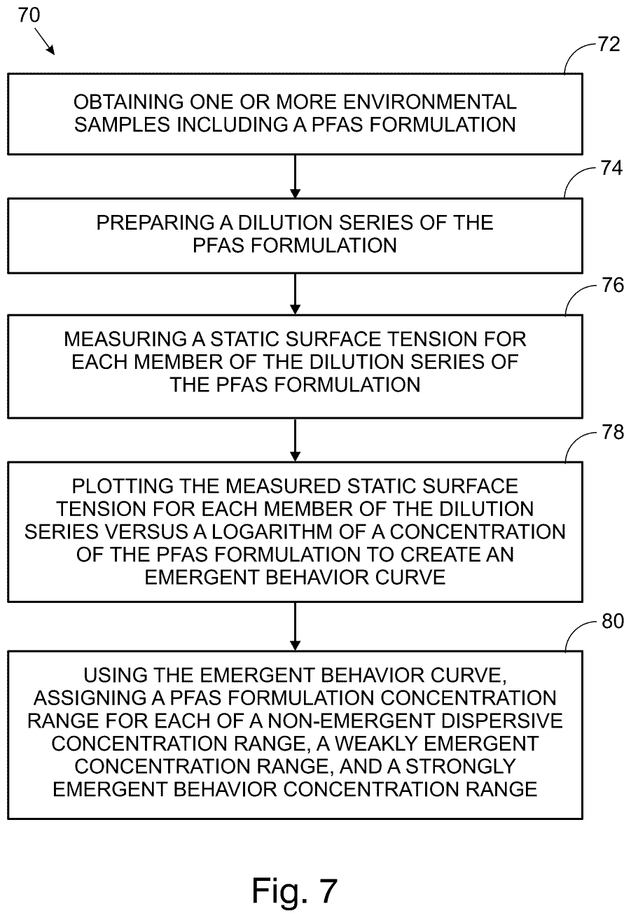

The analysis of the emergent behavior of PFAS-stabilized formulations lends itself to an accurate and advantageous method of characterizing a PFAS formulation, as set out in flowchart 70 of FIG. 7, including obtaining one or more samples including the PFAS formulation, at step 72 of flowchart 70; preparing a dilution series of the PFAS formulation; at step 74 of flowchart 70; determining a static surface tension for each member of the dilution series of the PFAS formulation at step 76 of flowchart 70; plotting the determined static surface tension for each member of the dilution series versus a logarithm of a concentration of the PFAS formulation to create an emergent behavior curve, at step 78 of flowchart 70; and using the emergent behavior curve, assigning a PFAS formulation concentration range for each of a non-emergent dispersive concentration range, a weakly emergent concentration range, and a strongly emergent behavior concentration range, at step 80 of flowchart 70.

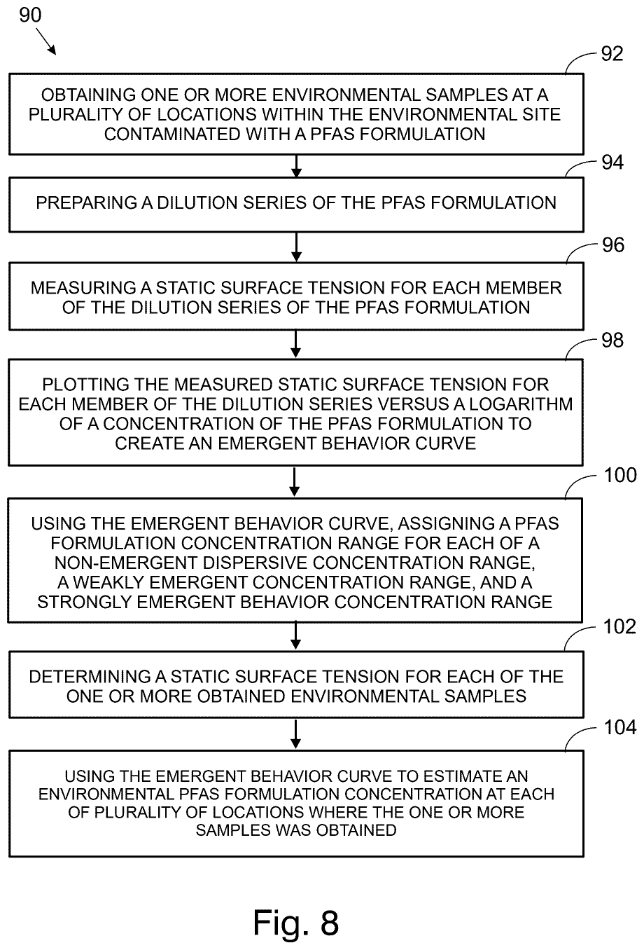

The analysis of the emergent behavior of PFAS-stabilized formulations additionally lends itself to an accurate and advantageous method of characterizing an environmental site contaminated with a PFAS formulation, as set out in flowchart 90 of FIG. 8, including obtaining one or more samples at a plurality of locations within the environmental site contaminated with the PFAS formulation, at step 92 of flowchart 90; preparing a dilution series of the PFAS formulation, at step 94 of flowchart 90; measuring a static surface tension for each member of the dilution series of the PFAS formulation, at step 96 of flowchart 90; plotting the measured static surface tension for each member of the dilution series versus a logarithm of a concentration of the PFAS formulation to create an emergent behavior curve, at step 98 of flowchart 90; using the emergent behavior curve, assigning a PFAS formulation concentration range for each of a non-emergent dispersive concentration range, a weakly emergent concentration range, and a strongly emergent behavior concentration range, at step 100 of flowchart 90; determining a static surface tension for each of the one or more obtained samples, at step 102 of flowchart 90; and using the emergent behavior curve to estimate an environmental PFAS formulation concentration at each of the plurality of locations where the one or more samples was obtained, at step 104 of flowchart 90.

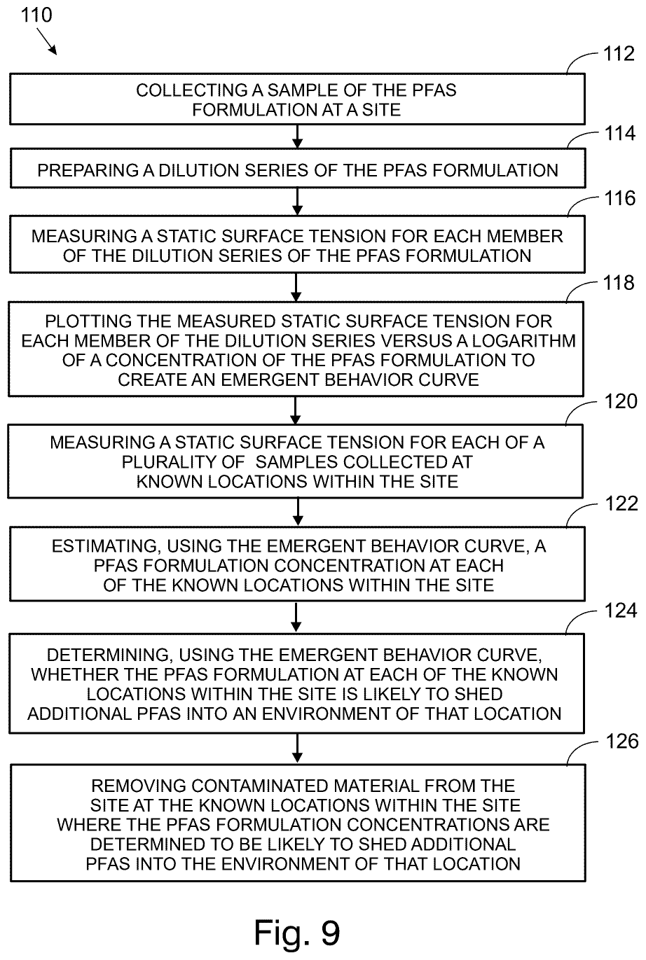

The analysis of the emergent behavior of PFAS-stabilized formulations additionally lends itself to an accurate and advantageous method of at least partially remediating a site that is contaminated with a PFAS formulation, as set out in flowchart 110 of FIG. 9, including collecting a sample of the PFAS formulation at the site, at step 112 of flowchart 110; preparing a dilution series of the PFAS formulation, at step 114 of flowchart 110; measuring a static surface tension for each member of the dilution series of the PFAS formulation, at step 116 of flowchart 110; plotting the measured static surface tension for each member of the dilution series versus a logarithm of a concentration of the PFAS formulation to create an emergent behavior curve, at step 118 of flowchart 110; measuring a static surface tension for each of a plurality of samples collected at known locations within the site contaminated with the PFAS formulation, at step 120 of flowchart 110; estimating, using the emergent behavior curve, a PFAS formulation concentration at each of the known locations within the site contaminated with the PFAS formulation, at step 122 of flowchart 110; determining, using the emergent behavior curve, whether the PFAS formulation at each of the known locations within the site contaminated with the PFAS formulation is likely to shed additional PFAS into an environment of that location, at step 124 of flowchart 110; and removing contaminated material from the site contaminated with the PFAS formulation at the known locations within the site where the PFAS formulation concentrations are determined to be likely to shed additional PFAS into the environment of that location, at step 126 of flowchart 110.

### Additional Selected Embodiments

This section describes additional aspects and features of the disclosed methods, presented without limitation as a series of paragraphs, some or all of which may be alphanumerically designated for clarity and efficiency. Each of these paragraphs can be combined with one or more other paragraphs, and/or with disclosure from elsewhere in this application, in any suitable manner. Some of the paragraphs below expressly refer to and further limit other paragraphs, providing without limitation examples of some of the suitable combinations.

Paragraph A1. A method of characterizing a PFAS formulation, including: obtaining one or more samples including a PFAS formulation; preparing a dilution series of the PFAS formulation; determining a static surface tension for each member of the dilution series of the PFAS formulation; plotting the determined static surface tension for each member of the dilution series versus a logarithm of a concentration of the PFAS formulation to create an emergent behavior curve; and using the emergent behavior curve, assigning a PFAS formulation concentration range for each of a non-emergent dispersive concentration range, a weakly emergent concentration range, and a strongly emergent behavior concentration range.

Paragraph A2. The method of paragraph A1, further comprising measuring a static surface tension for each of the one or more samples, and by comparing the static surface tension to the emergent behavior curve determining a concentration of the PFAS formulation in each of the one or more samples.

Paragraph A3. The method of paragraph A1, where the one or more samples are obtained from an environmental site that is contaminated by the PFAS formulation.

Paragraph A4. The method of paragraph A3, further comprising using the emergent behavior curve to correlate the determined concentration of the PFAS formulation in each of the one or more samples with a probability that the PFAS formulation for each environmental sample will shed additional PFAS at the environmental site.

Paragraph B1. A method of characterizing an environmental site contaminated with a PFAS formulation, comprising: obtaining one or more samples at a plurality of locations within the environmental site contaminated with the PFAS formulation; preparing a dilution series of the PFAS formulation; measuring a static surface tension for each member of the dilution series of the PFAS formulation; plotting the measured static surface tension for each member of the dilution series versus a logarithm of a concentration of the PFAS formulation to create an emergent behavior curve; using the emergent behavior curve, assigning a PFAS formulation concentration range for each of a non-emergent dispersive concentration range, a weakly emergent concentration range, and a strongly emergent behavior concentration range; determining a static surface tension for each of the one or more obtained samples; and using the emergent behavior curve to estimate an environmental PFAS formulation concentration at each of the plurality of locations where the one or more samples was obtained.

Paragraph B2. The method of paragraph B1, further comprising creating a map of the environmental site contaminated with the PFAS formulation showing which of the plurality of locations where the one or more samples was obtained includes an environmental PFAS formulation concentration that is within the non-emergent dispersive concentration range, includes an environmental PFAS formulation concentration that is within the weakly emergent concentration range, and includes an environmental PFAS formulation concentration that is within the strongly emergent behavior concentration range.

Paragraph B3. The method of paragraph B2, wherein the map of the environmental site includes location data in three dimensions.

Paragraph B4. The method of paragraph B1, where each of the plurality of environmental samples includes a water sample from the environmental site contaminated with the PFAS formulation.

Paragraph B5. The method of paragraph B1, where each of the plurality of environmental samples is prepared by a water extraction of a solid or semi-solid sample from the environmental site contaminated with the PFAS formulation.

Paragraph B6. The method of paragraph B1, further comprising using the emergent behavior curve to estimate a total oxidizable precursor concentration of the environmental PFAS formulation at each of the plurality of locations where the one or more samples was obtained.

Paragraph B7. The method of paragraph B1, further comprising correlating, using the emergent behavior curve, the estimated PFAS formulation concentration at each of the plurality of locations where the one or more samples was obtained with a probability that the PFAS formulation contaminant at each of the plurality of locations will shed additional PFAS into an environment of that location.

Paragraph C1. A method of at least partially remediating a site contaminated with a PFAS formulation, comprising: collecting a sample of the PFAS formulation at the site; preparing a dilution series of the PFAS formulation; measuring a static surface tension for each member of the dilution series of the PFAS formulation; plotting the measured static surface tension for each member of the dilution series versus a logarithm of a concentration of the PFAS formulation to create an emergent behavior curve; measuring a static surface tension for each of a plurality of samples collected at known locations within the site contaminated with the PFAS formulation; estimating, using the emergent behavior curve, a PFAS formulation concentration at each of the known locations within the site contaminated with the PFAS formulation; determining, using the emergent behavior curve, whether the PFAS formulation at each of the known locations within the site contaminated with the PFAS formulation is likely to shed additional PFAS into an environment of that location; and removing contaminated material from the site contaminated with the PFAS formulation at the known locations within the site where the environmental PFAS formulation concentrations are determined to be likely to shed additional PFAS into the environment of that location.

Paragraph C2. The method of paragraph C1, further comprising treating the removed contaminated material to remove the PFAS formulation until a static surface tension measurement for an aqueous extract of the treated material correlates with a PFAS formulation concentration in the treated material that is within the non-emergent dispersive concentration range.

Paragraph C3. The method of paragraph C2, further comprising transporting the treated material to a non-hazardous waste disposal site.

Paragraph C4. The method of paragraph C2, wherein treating the removed contaminated material includes removing the PFAS formulation from the removed contaminated material using thermal volatilization via a heated gas flow.

Paragraph C5. The method of paragraph C1, further comprising determining a total oxidizable precursor concentration for the PFAS formulation concentration at each of the known locations within the site contaminated by the PFAS formulation.

Paragraph C6. The method of paragraph C1, wherein each of the plurality of samples is a water sample from the site contaminated with the PFAS formulation, or is prepared by a water extraction of a solid or semi-solid sample from the site contaminated with the PFAS formulation.

### Advantageous Applications

The strategies and methods disclosed herein for characterizing and evaluating PFAS in PFAS-contaminated materials offer substantial advantages in a variety of applications, including but not limited to:
Guiding the course of investigations into potential PFAS contamination;
Providing data for the preparation of an accurate conceptual site model (CSM) for a contaminated or potentially contaminated site. A conceptual site model includes a written or graphical representation that encompasses the current understanding of a contaminated or potentially contaminated property, and provides a valuable decision-making tool for remediation planning, data interpretation, and effective communication for both technical and non-technical stakeholders;
Identifying and defining emergent PFAS source structures that are actively shedding PFAS into the environment;
Identifying areas where PFAS is actively moving downward through the soil column (vertical migration) and providing estimates for the rate of migration;
Determining a boundary for the establishment of an interim remedial action measure (IRAM) or remedial action to facilitate a transformation of the PFAS formulation concentrations present to a passive (non-emergent dispersive) status;
Determining a boundary for CERCLA (Superfund) divisibility;
Differentiating the presence of non-emergent dispersive PFAS sources from more concentrated emergent PFAS sources, that will continue to shed additional PFAS; and
Classifying and managing PFAS-containing waste under the Resource Conservation and Recovery Act (RCRA), for example by facilitating the development of an RCRA universal treatment standard (UTS) in conjunction with RCRA corrective action management units.
The emergent behavior curve may be used to assemble general response actions and technologies for optimal PFAS removal.

### CONCLUSION

As used herein and in the claims, “substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” may be used to distinguish or identify various members of a group, or the like, and are not intended to show serial or numerical limitation.

Unless they relate to specific examples, all specifications regarding quantities and portions, particularly those for delimiting the invention, indicate a ±10% tolerance, for example: 11% means: from 9.9% to 12.1%. For terms such as “a solvent”, the word “a” is not to be regarded as a numerical word but as an indefinite article or as a pronoun, unless the context indicates otherwise.

Although the present invention has been shown and described with reference to the foregoing operational principles and preferred embodiments, it will be apparent to those skilled in the art that various changes in form and detail may be made without departing from the spirit and scope of the invention. The present invention is intended to embrace all such alternatives, modifications and variances that fall within the scope of the appended claims.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, where the claims recite “a” or “a first” element or the equivalent thereof, such claims should be understood to include incorporation of one or more such elements, neither requiring nor excluding two or more such elements.

Inventions embodied in various combinations and subcombinations of features, functions, elements, and/or properties may be claimed through presentation of new claims in a related application. Such new claims, whether they are directed to a different invention or directed to the same invention, whether different, broader, narrower or equal in scope to the original claims, are also regarded as included within the subject matter of the inventions of the present disclosure.

What is claimed is:

1. A method of characterizing a PFAS formulation, comprising:
obtaining one or more samples including a PFAS formulation;
preparing a dilution series of the PFAS formulation;
measuring a static surface tension for each member of the dilution series of the PFAS formulation;
plotting the measured static surface tension for each member of the dilution series versus a logarithm of a concentration of the PFAS formulation to create an emergent behavior curve; and
using the emergent behavior curve, assigning a PFAS formulation concentration range for each of a non-emergent dispersive concentration range, a weakly emergent concentration range, and a strongly emergent behavior concentration range.

2. The method of claim 1, further comprising measuring a static surface tension for each of the one or more samples, and by comparing the static surface tension to the emergent behavior curve determining a concentration of the PFAS formulation in each of the one or more samples.

3. The method of claim 1, wherein the one or more samples are obtained from an environmental site that is contaminated by the PFAS formulation.

4. The method of claim 3, further comprising using the emergent behavior curve to correlate the determined concentration of the PFAS formulation in each of the one or more samples with a probability that the PFAS formulation for each environmental sample will shed additional PFAS at the environmental site.

5. A method of characterizing an environmental site contaminated with a PFAS formulation, comprising:
obtaining one or more samples at a plurality of locations within the environmental site contaminated with the PFAS formulation;
preparing a dilution series of the PFAS formulation;
measuring a static surface tension for each member of the dilution series of the PFAS formulation;
plotting the measured static surface tension for each member of the dilution series versus a logarithm of a concentration of the PFAS formulation to create an emergent behavior curve;
using the emergent behavior curve, assigning a PFAS formulation concentration range for each of a non-emergent dispersive concentration range, a weakly emergent concentration range, and a strongly emergent behavior concentration range;
measuring a static surface tension for each of the one or more obtained samples; and
using the emergent behavior curve to estimate an environmental PFAS formulation concentration at each of the plurality of locations where the one or more samples was obtained.

6. The method of claim 5, further comprising creating a map of the environmental site contaminated with the PFAS formulation showing which of the plurality of locations where the one or more samples was obtained includes an environmental PFAS formulation concentration that is within the non-emergent dispersive concentration range, includes an environmental PFAS formulation concentration that is within the weakly emergent concentration range, and includes an environmental PFAS formulation concentration that is within the strongly emergent behavior concentration range.

7. The method of claim 6, wherein the map of the environmental site includes location data in three dimensions.

8. The method of claim 5, wherein each of the plurality of environmental samples includes a water sample from the environmental site contaminated with the PFAS formulation.

9. The method of claim 5, wherein each of the plurality of environmental samples is prepared by a water extraction of a solid or semi-solid sample from the environmental site contaminated with the PFAS formulation.

10. The method of claim 5, further comprising using the emergent behavior curve to estimate a total oxidizable precursor concentration of the environmental PFAS formulation at each of the plurality of locations where the one or more samples was obtained.

11. The method of claim 5, further comprising correlating, using the emergent behavior curve, the estimated PFAS formulation concentration at each of the plurality of locations where the one or more samples was obtained with a probability that the PFAS formulation contaminant at each of the plurality of locations will shed additional PFAS into an environment of that location.

12. A method of at least partially remediating a site contaminated with a PFAS formulation, comprising:
collecting a sample of the PFAS formulation at the site;
preparing a dilution series of the PFAS formulation;
measuring a static surface tension for each member of the dilution series of the PFAS formulation;
plotting the measured static surface tension for each member of the dilution series versus a logarithm of a concentration of the PFAS formulation to create an emergent behavior curve;
measuring a static surface tension for each of a plurality of samples collected at known locations within the site contaminated with the PFAS formulation;
estimating, using the emergent behavior curve, a PFAS formulation concentration at each of the known locations within the site contaminated with the PFAS formulation;
determining, using the emergent behavior curve, whether the PFAS formulation at each of the known locations within the site contaminated with the PFAS formulation is likely to shed additional PFAS into an environment of that location; and
removing contaminated material from the site contaminated with the PFAS formulation at the known locations within the site where the PFAS formulation concentrations are determined to be likely to shed additional PFAS into the environment of that location.

13. The method of claim 12, further comprising treating the removed contaminated material to remove the PFAS formulation until a static surface tension measurement for an aqueous extract of the treated material correlates with a PFAS formulation concentration in the treated material that is within a non-emergent dispersive concentration range.

14. The method of claim 13, further comprising transporting the treated material to a non-hazardous waste disposal site.

15. The method of claim 13, wherein treating the removed contaminated material includes removing the PFAS formulation from the removed contaminated material using thermal volatilization via a heated gas flow.

16. The method of claim 12, further comprising determining a total oxidizable precursor concentration for the PFAS formulation concentration at each of the known locations within the site contaminated by the PFAS formulation.

17. The method of claim 12, wherein each of the plurality of samples is a water sample from the site contaminated with the PFAS formulation, or is prepared by a water extraction of a solid or semi-solid sample from the site contaminated with the PFAS formulation.
